(19) United States
(12) Patent Application Publication
(10) Pub. No.: US 2014/0148642 A1
(43) Pub. Date: May 29, 2014

(54) Magic genie developer

(71) Applicant: Gene C. Benckini, Coopersburg, PA (US)

(72) Inventor: Gene C. Benckini, Coopersburg, PA (US)

(21) Appl. No.: 12/930,512

(22) Filed: Jan. 10, 2011

Related U.S. Application Data
(60) Provisional application No. 61/338,666, filed on Feb. 22, 2010.

(51) Int. Cl. A61F 5/41

(52) U.S. Cl. CPC A61F 5/41
USPC 600/38; 424/94.63

(57) ABSTRACT

Apparatus and formulations for correcting and curing erectile dysfunction in men to create a natural erection without the use of primitive devices. the formulations will correct and cure erectile dysfunction along with the vacuum apparatus to aid the correction and cure, but also enhance the size of the penis AFTER clearing out the plaque build-up and improve circulation to the male organ, by the use of the elongated cylinder to insert the penis into the plastic portal with the pulsating action with the other end closed, except for the intake vacuum pressure to help the formulations process to stimulate the penis and also the cleaning of arteries and vein build-up of plaque where the problem originally started to prevent the natural erection without any or poor blood circulation, which was the initial blockage in the arteries and veins in the corpus cavernosum and corpus sponglosum chambers the now formulations of #501 and #503 will work to clear up the circulation problems for ever and #502 will enhance the overall length and girth greater then prior erections.

### REFERENCES TO SPECIFIC PROBLEMS INVOLVED IN THE PRIOR ART OF INVENTIONS

All (25) listed prior art inventions have failed to address the major problems with impotence and erectile dysfunction by only presenting an artifical erection and only a (temporary solution) to the problems associated with over 10,000,000 men who suffer on a day to day bases, this is not a psychological problem, but a physical problem with circulation of the males blood flow to the penis through the main arteries and veins, americans are the real problem with what they eat, and with all the junk foods on markets and fast food restaurants with processing with lard and cheap frying oils, we are living in a junk food serving world, over 25/50 years of this, common sense will tell you people are dieing of heart attacks, this plaque over years is clogging the arteries and veins which is clearly associated with the blood flow to the penis, the penis is shrinking, the veins are shrinking, but men have associated this problem with age, which is totally wrong, the answer to cure and rejuvenate the erection of the penis is within reach, associated with a formulation to clean out the main arteries and veins, this is a wake-up call, people must educate them selfs, its ridiculous to go through life with out a natural erection and gratification of sex, these prior art only touch the surface of what could be reality with the correct formulation and the best invention ever to ever solve the problems associated with impotence and erectile dysfunction (the magic genie developer).

Yes, thousands of men out of desperation to have sex paid a high price for surgery, steroid injections and many men ending up with injuries and totally disappointments, I have talked to many men over the last (10) years who complain about herbal formulas that do not work, but I know different, I realized that their problem is internal and not external.

Here within is a basic internal structure of how the blood should flow for a natural erection, the dorsal artery which lays on top of the penis and the deep dorsal vein that that charges the corpora cavemosa which is like a sponge, the bulbourethral artery and vein on the bottom of the penis feed the corpus spongiosum to expand the penis into a good erection for performance in the desired sex act, this would of course be a natural reaction for a healthy penis, but on the other hand with clogged arteries and veins feeding the penis at only 35/50% of the required blood pressure, sure the male wants to have sex, but the equipment is malfunctioning and the result is (mission impossible) it take years of sloppy eating habits and plain old ignorance, to line your arteries and veins with plaque, the average male will associate this problem with age or psychologival reasoning and most men are living with this condition and out of desperation buying these worthless hand operated penis pumps and some with constrictor rings, but you must realize as soon as the blood pressure from these artifical erections is released the penis becomes flaccid again and if the user keeps the ring on the base of his pressure penis, how in hell is this poor sucker going to ejaculate, I'm sorry, I could not imagine trying to have this really exciting restricted, it must be painful and depressing these prior art inventions do not solve the inital internal problems and only prolong the problem, do you realize that this problem in a marriage will lead to divorce or betrayal of his partner, and for what, when the problem is clearly within reach with a little common sense. Yes, I'm 71 years of age, yes I was a victim of this disorder for over (10) years I got soon mad at being worthless, that I stopped dating, sure, every women I knew wanted sex, and I was fairly hung, but I took notice my penis started to shrink and my penis veins were shrinking away, all I used my precious cock for was to urinate with, really sad, sad, sad, I started making excuses to cover up the problem, I would get so depressed that I punched a hole in the door, kicked out panels in walls, this really was insane, I was desperate, I started to do lots of research and experimenting over (10) years and now boys I'm proud to say the least with my formulations and invention (magic genie developer).

There must be well over (50 inventions) out there based on the same principles, vacuum pressure in a cylinder to pull a dead an lifeless penis into sucking the blood into the penis, for what, the real problem has not been solved, only prolonged for over 10,000,000 men who still suffer, well I want to come to the aid of these men, because I know how difficult life can be when a man has impotence and or erectile dysfunction.

(I will list the prior art of these inventions here refered to)

In my (3) page references to specific problems which these prior inventions have failed to accomplish.

U.S. Patent Documents:

| | | | |
|---|---|---|---|
| (1) | BDES-421,652 | Mar. 14, 2000 | STEVEN C. GAMPER |
| (2) | 2001/0044569 | Nov. 22, 2001 | MONSHI NAGHAR |
| (3) | 4,175,554 | Nov. 27, 1979 | FRANK J. GEROW |
| (4) | 4,378,008 | Mar. 29, 1983 | GEDDINGS D. OSBON, SR. |
| (5) | 5,095,895 | Mar. 17, 1992 | MICHAEL W. WALSH |
| (6) | 5,125,890 | Jun. 30, 1992 | DANIEL C. MERRILL - BAD NEWS. |
| (7) | 5,951,460 | Sep. 14, 1999 | ANDREW J. VOLLRATH |
| (8) | 6,277,062 B1 | Aug. 21, 2001 | ANDREW J. WOLLRATH _VERY COMPLEX. |
| (9) | 7,186,213-B2 | Mar. 6, 2007 | JULIAN OSBON |
| (10) | EP0148586 A | Jul. 17, 1985 | NADIG W. PERRY |
| (11) | 4,378,008 | Mar. 29, 1983 | GEDDINGS D. OSBON, SR. |
| (12) | 3,744,486 | Jul. 10, 1973 | ELDON M. WILSON |
| (13) | 4,671,262 | Jun. 9, 1987 | BOYCE W. WEST |
| (14) | 5,234,401 | Aug. 10, 1993 | HIDEO YAMANAKA |
| (15) | 5,782,621 | Jul. 21, 1998 | JESSE W. HARRIS |
| (16) | 5,421,808 | Jun. 6.1995 | JAMES B. OSBON |
| (17) | 4,741,329 | May 3, 1988 | BENJAMIN F. MARCUME |
| (18) | 5,836,864 | Nov. 17, 1998 | ROLAND T. CLARK, JR. |
| (19) | 4,175,554 | Nov. 27.1979 | FRANK J. GEROW |
| (20) | 4,718,411 | !/12/1988 | EDWARD T. STEWART |
| (21) | 3,631,853 | Jan. 4, 1972 | MARVIN A. BURDETTE, JR. |
| (22) | 5,115,800 | May 26, 1992 | NENAD MATEJEVIC |
| (23) | 4,856,499 | Aug. 15, 1989 | EDWARD C. KELLY |
| (24) | 4,753,227 | Jun. 28, 1988 | RUDOLPH R. YANUCK, JR. |
| (25) | 4,856,498 | Aug. 15, 1989 | JULIAN W. OSBON |

(INT.Cl.) A61F 5/00
(US Cl) -------------------- 600/38
(FIELD OF SEARCH ------------ 128,897,899,417,550,544,306.

of all these prior art inventions are basicly using the same principles, either manual or battery operated vacuum pumps, and some with constrictor rings which I feel are risky to use.

Not one of these inventions deal with the internal problems impotence and erectile dysfunction for cure and correction to solve the initial blockage and circulation of the arteries and veins with plaque, of course a man can not achieve an erection if the blood pressure is limited to 35/50% of flow, thats like trying to start an engine without gas, these prior art inventions are just spinning their wheels and failing to correct the initial reason that men can not achieve a natural erection, we are in the age of modern technology not the stone age, lets move on and help these 10,000,000 men who suffer from impotence and erectile dysfunction, which is actually ruining these men's life, their marriages, jobs, attitude at work, low self-esteem, embarrassment, positive direction of his future, all these pit-falls reflect on this suffering male, (a man must be a man and that includes getting a natural erection) not a temporary solution and ongoing problem. Believe me I know I have been down this road of depression and despair, I was personally sick of the entire problem and I not only cured and corrected my problem, but I ended up getting the longest and thickest penis that I ever had, I'm proud of it.

### BRIEF SUMMARY OF THE INVENTION

(1) The inventor created this invention system for himself of course the inventor was inflicted with the condition of impotence and erectile dysfunction for years before he was able to perfect a formula #501, 502, and 503 to cure and be able to correct the male genital malfunction on his own.

(2) Formula #501 was created to clean the veins and arteries of the male genital organ so as to start functioning normally, the #502, the magic genie developer was designed to help and in conjunction with formula #501 to place vacuum on the organ with a clear plastic cylinder with the idea of expanding the arteries and veins while slowly forcing blood through the structure of the male organ and it worked well, the organ had become much bigger in length aim girth, but with no spine, it was thought that since the genital organ was dormant stage for years and needed to be rebuild with the idea of the formula #503 to build-up the nitric oxide levels and testosterone to regain the strength and endurence needed to maintain a natural erection without useless manual pumps and constricter rings to force the blood to stay in the erect penis, but they, the prior art all failed to address the problem directly and cure the malfunction.

(3) This invention system has many advantages over the (25) prior art documents of their inventions, first its electrical and user friendly, no vacuum gages, no pressure control valves to keep monitoring, its totally automatic with the internally controlled mechanism with preset vacuum pressure that never changes, its mounded on four rubber booties to keep it very quiet it has a therapeutic pulsating action directed to the clear plastic cylinder to stimulate the male genital organ, which none of the prior art designs possess and its segregated from all of the prior art documents listed as reference to and not one of these inventions address the major medical malfunction its sad, sad to examine these prior inventions and not see any cure or correction for impotence or erectile dysfunction for the over 10,000,000 men, I feel sorry for them as I felt for myself in this mental depression and dispair, my foundest wish would be to know that if I could cure and correct myself then at least some of these poor soles will be cured.

I'm personally proud to have contributed to humanity in some way that would help these men on the road to recovery.

### FIG. (1)

### The Magic Genie Developer

### Description

(1) The main body housing which operates without any lubrication ever and functions by electricity which activates the two internal magnetics.

(2) The duel vacuum valve housing units, that are activated by the both magnetic plates internally set to control the vacuum pressure with the intricate mechanism.

(3) The diaphragm vacuum pumps duel intake lines on both sides to synchronize the vacuum pressure evenly.

(4) The T-bar connector for the duel vacuum lines, which is screwed to the diaphragm housing.

(5) The threaded metal nipple connecting the T-bar to the adapter of the plastic cylinder backing plate.

(6) The double threaded female adapter connecting to the plastic cylinder's backing plate for a secure fitting.

(7) The nine and one half elongated cylinder of transparent type plastic with a one sixteenth inch wall for durability never to break and internally smooth surface to receive the male organ for the development process.

(8) The smooth portal double roll-over flang for safety and easy one and seven eighth inch inside dimensions to three inch inside dimensions for various male organ sizes in a flaccid stage for the development process of the male organ.

(9) The six foot electrical extention cord for the operating convenience of the user with a ground lead.

(10) The inline electrical on/off switch for the users within hands reach of the diaphragm pump.

(11) The three way electrical plug for ground lead outlets.

(12) The main diaphragm vacuum pump housing with four bolted rubber booties to control and direct the building pulsation action directly to the elongated cylinder which in turn stimulates the development process of the male organ.

(13) The four bolts on each corner of the vacuum valve housing to stabilize and quiet the pulsation action.

### FIG. (2)

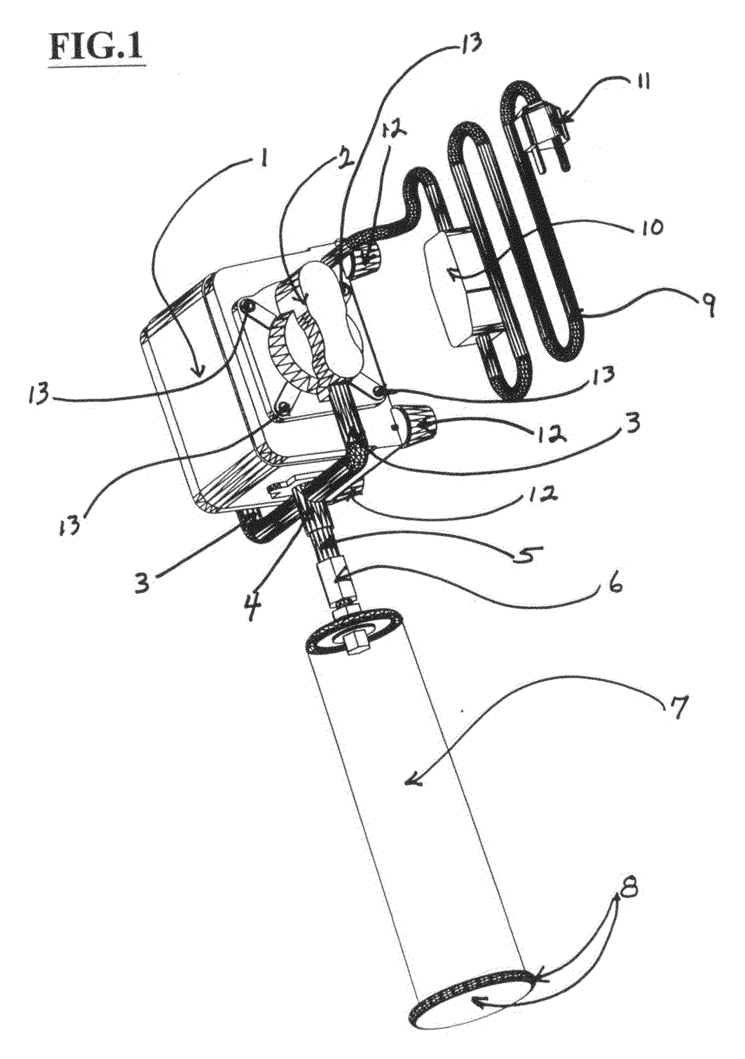

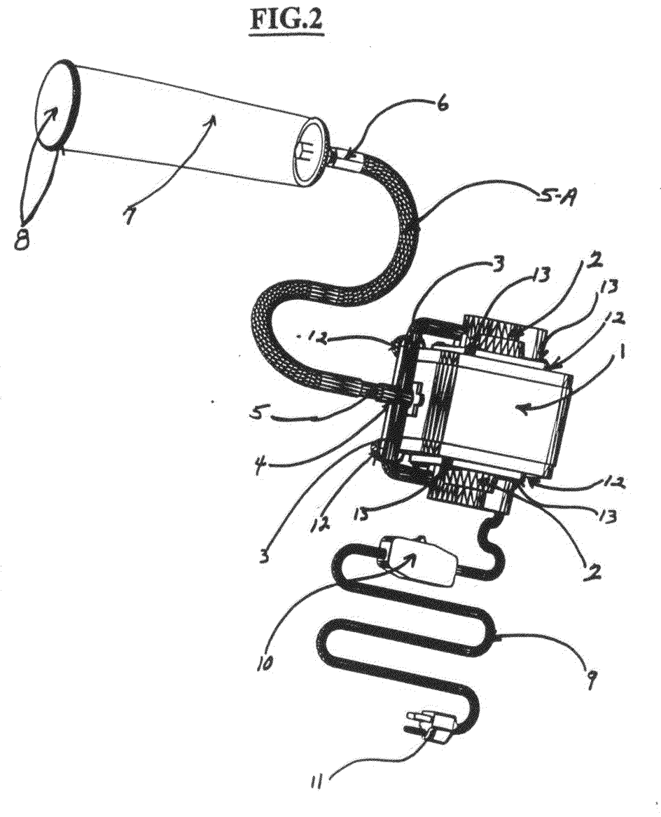

The Magic Genie Developer is basically the same as FIG. 1 except for the number (5-A) six foot long one half inch flexible plastic vacuum hose for the convenience of the user to place the diaphragm vacuum pump in or on a cabinet or desh drawer.

(5-A) One six foot extention one half inch vacuum hose line for the convenience of the user to use the diaphragm pumps extention hose line to use it in more privacy.

Also the magic genie developer unit may be placed in the basement of a home in a cabinet with space and run a one half inch plastic rigid line across the ceiling and up through the floor to a private room for convenience and privacy of use without ever hearing the vacuum pump operating and also there is no lost of vacuum pressure which remains precisely the same through the entire development of the male organ, this feature is buildin with the electrical activated magnetic plates.

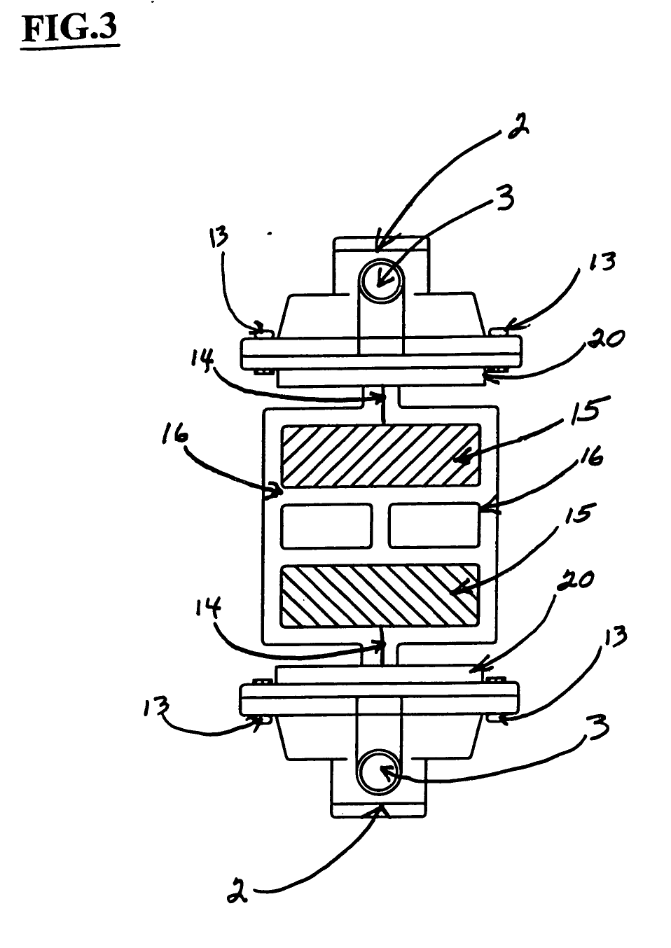

### FIG. 3

### The Magic Genie Developers Internal Mechanisms

### Intricate Description

(2) The duel air valve housings which contain two separate vacuum valves of precisely the same vacuum air pressure.

(3) The valve portals of the vacuum air pressure to receive the air connecting lines on both sides directly to the #14-T-bar connector which is bolted to the center of the diaphragm pump housing to stabilize the vacuum lines.

(13) The bolts on all four corners of both duel valve housings to stabilize and seal off the low noise level.

(14) The duel electrically activated ⅛″ steel rods that are connected to the internal magnetic plates that are forced to move at a rapid pace, but in only ¼″ movements to create the internal vacuum air pressure.

(15) The duel heavy duty magnetic plates wrapped in very fine coiled electrical wire which is an intricate part of the of the vacuum air stabilizing feature which regulates the air vacuum pressure to both valves.

(16) The magnetic plate housing that is molded around the both of the magnetics with the fine coiled electrical wire and supports the #14 steel rods fast and short movement at a rapid pace.

(20) The diaphragm backing plate that bolts to the vacuum housing and incorporates the both soft rubber membranes that are synchronize the intake of vacuum pressure of air that never changes.

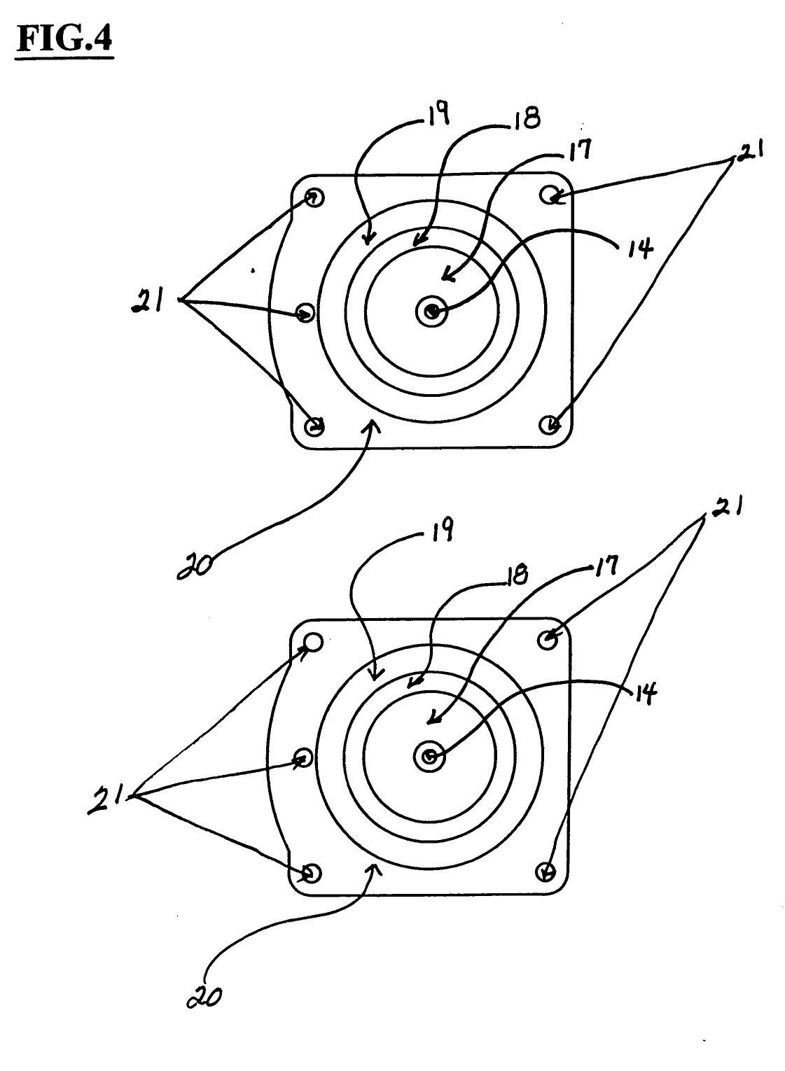

### FIG. 4

### The Magic Genie Developer's Membrane Housing

(14) The ⅛″ steel rods that operate the internal and external membranes and valves.

(17) The plastic bolted disk that covers and supports the rubber menbranes of the duel vacuum air intake created by the very rapid movements of the steel rods connected to the magnetics

(18) The duel rubber disk like membranes that are fastened and supported by #17 the plastic bolted disk.

(19) The duel backing plates fastened to the plastic disks of #17 that hold and support the soft rubber membranes that control the vacuum air pressure.

(20) The diaphragms duel backing plates that bolt to the vacuum housing that incorporates the #14 steel rods and the #17 plastic disks, with #18 the rubber membranes and #19 the backing plates of the membranes mechanisms.

(21) The four bolt holes on the duel backing plates that are used for #13 bolts to fasten to the vacuum valve housings.

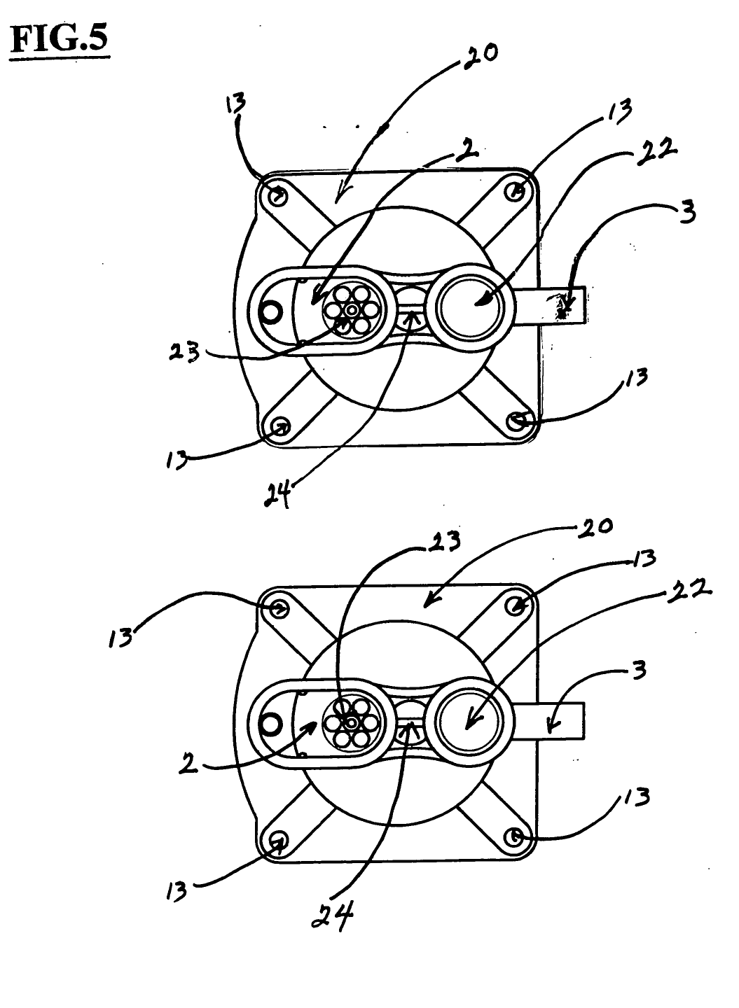

### FIG. 5

### The Magic Genie Developers Housing of Valves and Membrane

### Note: As Numbered on the FIG. 5

(2) The duel valve housings on each side of the diaphragm vacuum pump.

(3) The duel vacuum intake air ports that connect to the duel vacuum lines of the diaphragm pump.

(13) The bolts on each of the four courners of the duel valve housings.

(20) The duel housings backing plates that incorporate the internal membrane and fastened to the main diaphgragm housing body.

(22) The duel vacuum air valves that synchronize the vacuum intake air on both sides to properly create the internally designed vacuum air pressure directed to FIG. 1 #8 the elongated cylinder's flanged port and the transparent plastic development cylinder to insert the male organ.

(23) The diaphragm's relief valve which maintains equal air vacuum pressure to the duel valves as a backup feature.

(24) The valve housing's filter insert that collects any dust and is also access to the adjusting nut on the steel rod of the pumping mechanism that operates the plastic disk and the rubber menbranes.

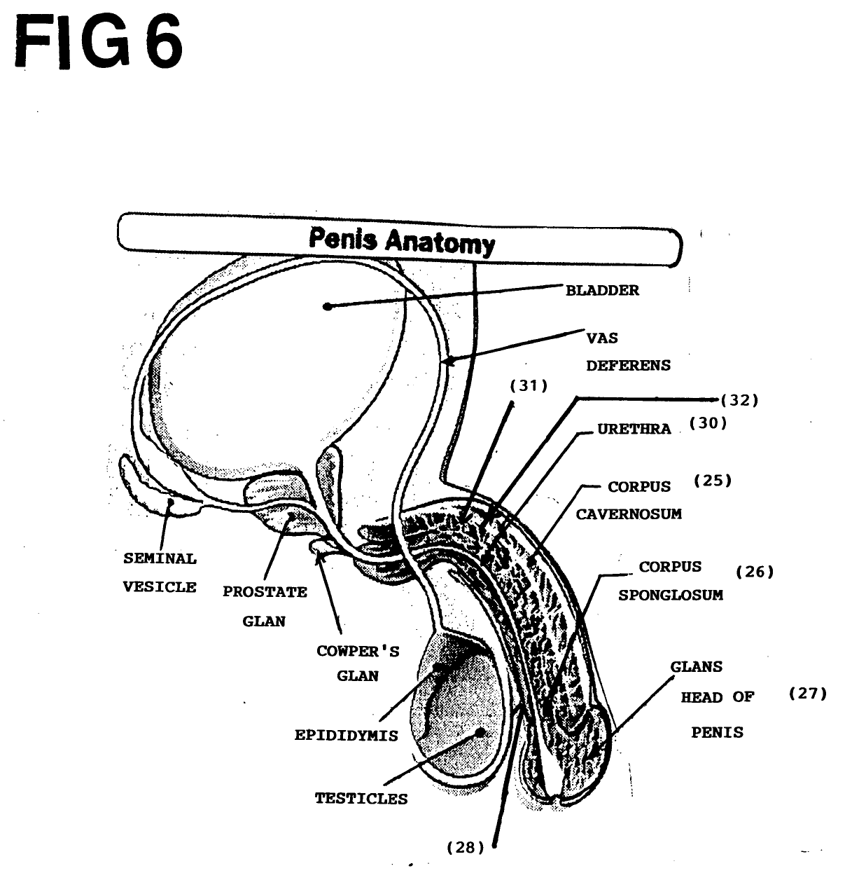

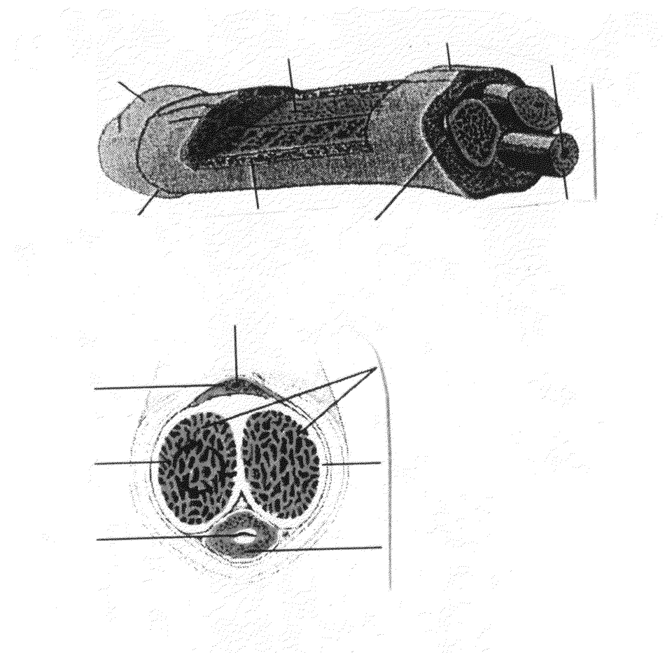

### FIG. 6

### The Internal Components of a Very Complex and Complicated Genitle Organ in Its Normal Stage of Erection

(25) The vast chambers of the corpora cavermosa and the corpus spongiosum with the upper and lower illustrated diagrams clearly shows thousands of cells and tissues that make-up the entire body which must function in harmony in order to achieve a full and natural erection.

(26) The corpus spongiosum also has its own chambers of cells and tissues that expand under normal blood pressure, but in reality and tests, experiments time after time have proven that yes the user will get the same size erection every time, simply because the user only has the same blood pressure to expand the cells and tissues to that limited degree, but proven tests with the (magic genie developer) has used precisely and exactly the right air vacuum pressure to bring out the genitle organs most potential degree of expansion to the corpora cavermosa and the corpus spongiosum and the glans penis head increasing the penis length and girth by 35/60% more of its original size before the treatment of the (magic genie developer).

(27) The glans penis head is liminted to the same normal size that was untill (the magic genie) stepped into the picture that may well double the size of the penis head for more sensitivity and more pleasure for the female in stimulating her vagina's canal, also the additional length and girth will place more pressure on the female's clitoris and of now with the increased length you may surge all the way up into the uterus means more climaxes.

(28) The outer shield of expansible skin which encases the corpora cavermosa and the corpus spongiosum and protects the cells and tissues from exterior use.

(29) The tunica albuginea is also a strong inter shield that expands like an inner-tube of a bike tire it also protects the complex genitle organ from injury and like the air in the inner-tube it expands with additional blood pressure to yield to a higher degree of expansion above and beyond.

(30) The uretha passage for the urinary tract to keep the passage open and the prostate glan healthy.

(31) The dorsal vein is very important to deliver blood to all the spider veins in the genitle organ that contribute to the harmony of the erection with the arteries.

(32) The dorsal artery rises and expands with the sexual excitement to feed the corpora cavermosa and the corpus spongiosum to the degree of vacuum pressure delivered.

### Formulation #501

### 30 Day Treatment for the Plaque Removal from the Arteries and Veins of the Male Organ

(1) 400 IU of vitamin (E) in the morning and one 400 IU vitamin (E) in the evening every day for (30 days) total 800 IU.

(2) Take two capsules of triple strength nattozimes 195 mg. In the morning & two capsules in the evening after meals. total of (4) capsules daily.

(3) Take two capsules of Horse chestnut, one in the morning and one in the evening, horse chestnut 200 mg. Only (2) for the entire day.

(Horse chestnut is a slow release)

(4) Take one to two table spoons of apple cider vinegar per day with meals and best of all, switch salad dressings from high cholesterol to a regular habit of apple cider vinegar (5% acidity) content with all salads the rest of your life, it will keep you healthy and great for the clearing the plaque in the arteries and veins is called (Pectin) which help prevent blood clots and heart attacks.

(5) Remember now to drink plenty of water using (#2) and (#3) formulations, a 120 lb. person should drink 600 oz. a day or a person 200 lbs. atleast 90 oz. of water per day.

I'm 71 years old this formulation that I created works well for me, the veins and arteries start to increase in size which means the formulation is working and you will feel more energy.

### Formulation #503

### For Rejuvenating the Entire Genital Organ in Creating a Natural and Increased Size Erections

(1) L-arinine 3/6 grams daily, 3000-g. morning and 3000 g. in the evening since the evening time is when the pituitary glan stores hormones that react directly to the sexual excitement of the male and then to the genitle organ of erection.

(2) Butea superba 500 mg. one in the morning and one in the evening after meals, used for rejuvenating the penis and also the human well being of strength and energy.

(3) Vitamin (E) 800 IU per day, one in the morning and one in the evening after meals at 400 IU per dosage.

(4) Catuaba bark 465 mg., (2) capsules in the morning and (2) capsules in the evening to help develope the size of the penis.

(5) Icariin extract 40%, 500 mg. twice a day, one in the morning and one in the evening. Great for erections and also body building of muscles and strength.

(6) Tongkat ali extract 1:200, 300 mg., two capsules in the morning and two in the evening, helps build the size of the penis and also for body building of muscles.

(7) Tribulus terrestris 500 mg. one capsule in the morning and one in the evening will help build blood circulation and also great for erections and body building of muscles.

(8) Zinc 75/100 mg. per day to help build up the nitrate oxide levels, no nitrate oxide, no erections.

(9) Last but not least, Velvet Deer Antler 500 mg. capsules take two capsules per day, one in the morning and one in the evening for a powerful rejuvenating effect for sexual functing, increase testosterone, high, and energy levels.

This formulation will slowly take over your life without you even thinking about it, just like a blood transfusion, your body will start to feel stronger more energy, more endurance more sex, your many aches and pains will slowly weaken and some will disappear, and funny as it seems your attitude will slowly change, you will be more considerate, more polite, and you will find yourself laughing to yourself, and smiling and instead you will find smiling to other people easier, it just takes over your life for well being and health another thing don't laugh you will start to look younger your complexion.

(you will see the difference in six months)

### INSTRUCTIONS

### For the Operation and Use of This Invention System of #502 the Magic Genie Developer

### Hands on User Friendly and Easy to Use

Take the new unit, The Magic Genie Developer out of the shipping box and place the diaphragm pump on a desk top take the electrical cord and plug it into the out-let and next take the long transparent cylinder out and then screw it into the threaded end of the pump, then take a amount of lotion with aloe vera in it and place a thin layer either on your penis or inside the cylinder about ⅘ inches and spread it around evenly, now your already to insert your flaccid or semi-erect penis into the entrance of the portal of the cylinder, now turn on the, Magic Genie Developer unit with the inline switch, the diaphragm pump will slowly pull your penis into the developer cylinder all the way in to the very base of your penis so a tight seal is made and you may watch the development process or read a book watch T.V. for a full (25/30 minutes of treatment, now you will turn off the unit and pull your huge penis out of the cylinder and wash your penis free of lotion, your penis will 35/65% longer and with much more girth.

Caution, you must not place your new penis in any tight under wear or shorts and no tight dress genes, because your penis development is on going for at least one hour and must be allowed to grow longer and thicker, also do not use the magic genie developer more then (3) treatments a week, you must allow your new found python rejuvenate its cells and tissues.

### BACKGROUND OF THE INVENTION

### How it Originated

The inventor was stricken with impotence and erectile dysfunction in 2001 and then researched and studied the problems associated with this disorder. I had the idea of creating a vacuum pump that could perhaps stimulate the penis into correcting it self.

I started with a low volume pressure pump and disassembled it when its original use was to blow low pressure air into aquariums I had converted the valves from air pressure out put to vacuum which only had 2,5 in hg., inorder to increase the vacuum pressure I pulled the coil out and kept adding wired revolutions of more wire untill I build the vacuum pressure up to (8.5 in hg.) then since it was operated by magnetics on either side I had increased the draw of the magnets and was ready to test the pump, of course when started with a ¼″ short line for vacuum, it had very little vacuum pressure on the tip of my finger, but when I had installed a (10″ long arcrylic clear tubing with 2″ ID.) which in turn created much more cubical volume of air pressure up to (8.5 in hg) vacuum, then I placed the limp penis into the cylinder using a little lotion and after (30 minutes of treatment) I extracted the penis, yes, it was longer and more girth, no doubt about that, but the penis had (no spine) just big and limp. Of course a man can not have intercourse with a limp penis. I started with ways to make the penis more rigid, instead of a balloon half full of air, (of course what was I thinking) no air in the balloon, no blood pressure in the penis, logical reasoning, right.

I did notice the penis had originally shrank and the veins had almost disappeared, my arteries and veins must be clogged with plaque build-up, after all I'm (71 years of age) I had ran the New York city marathon in 1987 and 1988, I felt good then, but time has its toll on man, I started doing research on (clog busters) and came up with my formula #501 which I will list on page (2) once I had created a good formula of clog busters I started the program with my formula #501 for one full month to clean out the arteries and veins, after 2/3 weeks of treatment I could feel my penis getting longer and fuller, the penis veins were much more vivid, I waited untill after (30 days) then I incorporated the step (2) process of my (magic genie developer) to help expand the penis cells and dorsal artery along with bulborurethral after which would inturn expand all of the penis veins, which would help pull more blood in to the arteries and veins, this process took (30 minutes of treatment) I could feel much more excitiment in the penis as it was being pulled into the (10″ cylinder) and after (30 minutes of treatment) I extracted a huge penis that had filled the arcylic (2″ ID. cylinder) the penis was over 60% erection and long and much more girth, I knew then I was on the right track for recovery, all I had to do was stay on the program, I was so thrilled that I cryed, the tears were rolling down my cheeks, I looked into the mirror and started to laugh, with tears, if you could understand my accomplishment, I was curing and correcting my impotence and erectile dysfunction, but still I needed step (3) formula #503 to boost the erection process and be able to obtain a natural erection when I needed it for action, the penis must by it self without any constrictors, no artifical erections, no trying to pull off the constrictor ring while in the excitiment mode with her begging for more.

Formula #503 build-up such a strong erection with the help of #502 the magic genie developer. I'm very proud that I have created a cure and correction for over 10,000,000 men inflicted with this disorder as I was, (this would be my contribution to man-kind, amen)

I will list the formula #501, the magic genie developer #502 formulation #503 on a separte page (3). Thank you.

1. this invention system with the formulations of #501 and #503 are systematic with #502 the vacuum system, the magic genie developer to cure and correct “erectile dysfunction” for a natural erection, not an artificial erection.

2. formulation #501 will help dissolve the plaque build-up over years of poor eating habits and open the arteries and spider veins of the penis to correct required blood flow to the penis and eliminate “erectile dysfunction” problems in the future.

3. the invention system #502 was designed with the proper and safe vacuum pressure with the very best natural results and greatly help the formulation #501 to increase the blood flow to the penis for better circulation of the main arteries and spider veins along with the aid of formulation #503 in creating and building a stronger erection with greater length and girth in a very natural erection to cure and correct erectile dysfunction for future sexual satisfaction.

4. the #502 invention system with a designed intricate mechanism to regulate the exact vacuum pressure with out any fluctuation to achieve the very best penis enlargement results, this unit has totally eliminated the problems with the #25 prior art inventions which require a pressure gage and valve to manually keep regulating the vacuum pressure back and forth, but the #502 vacuum system also completely eliminated the dangerous restrictor rings that prevent any possibility of any ejaculation release that would be painful and unhealthy, also the strictor rings are only designed for an artificial erection for no more then 30 minutes regardless if the strictor ring design is with battery or hand operated pumps, these inventions are obsolete.

5. the invention system #502 has an amazing test results in combination with formulation #501 and formulation #503 that will almost double the size of the penis in length and girth, the formulation combination has a great effect on the penis anatomy, first off the corpus cavernosum and the corpus sponglosum are greatly stimulated by the formulation #503 combination of herbs over a period of 10 days use and the direct use of the invention system “the magic genie developer” #502 will form the penis into the user's imagination come to life, at least ⅘″ in length and ¾″ in girth, also the users testicles will increase in size and the users ejculations will be much stronger and fuller, this increased additional ⅘″ in length and ¾″ in girth will provide a greater stimulation for the female's vagina and clitoris which will result in greater orgasms for her, she will be knocking down your door for more.

6. this invention system #502 has a build in designed pulsation action to help with the developement of the penis size, and the elongated cylinder is transparent with a double roll over flang for smooth vacuum and sealing off the air pressure for the best results.

7. this invention system #502 is user friendly that even a blind man could operate it, also the the vacuum pump maybe left positioned to the elongated cylinder and stored in that manner, the vacuum system comes with a 6′ long ½″ plastic flexible air hose for more convenience to the user, also the electric cord has a buildin shut off switch for convenience.

8. in this world of over (5) million men suffering from “erectile dysfunction” and penis shrinking penis size this major physical problem is now within reach with the formulation #501 and # 503 combination of herbs, the log here is very low blood pressure and circulation blockage with contributing low nitrate oxide levels that mostly come with poor eating habits and older age, when there is very low blood circulation a male finds it impossible to obtain an erection, formulation will help solve that Problem, the low level of nitrate oxide is a major reason men can not achieve an erection, but with the formulation #501 and #503 combinations of selected herbs there is an answer to this problem, the arteries must be opened up to allow more circulation of blood, which #501 may greatly help and once that is achieved then the #503 formulation of selected herbs go to work on restructing the the penis anatomy of the corpus cavernosum and the corpus sponglosum with vital herbs to rejuvenate the cells of this organ that has been in a negligent stage for years without realizing the internal problems with simple logic.

9. in all (25) prior at inventions, not one person has ever investigated the internal problems, prior art only has addressed the external application of useless apparatus to never solve the internal problem which is really in sight with common sense and logic, there are millions of men with erectile dysfunction who do not know where to turn untill now, we can help with the cure and correction of “erectile dysfunction ” with our formulations of #501 and #503 in conjunction with our invention #502 “the magic genie developer” will not only cure and correct “erectile dysfunction” but our #502 will give the user a greater length and girth of his penis then ever imaged before with results within the first thirty minutes of use, no less then 2″ in length and 2″ in the girth.
